(19) United States
(12) Patent Application Publication
(10) Pub. No.: US 2012/0064563 A1
(43) Pub. Date: Mar. 15, 2012

(54) CELL-BASED SENSING SYSTEMS AND METHODS

(76) Inventors: Alborz MAHDAVI, PASADENA, CA (US); Hua WANG, CAMPBELL, CA (US); David A. Tirrell, PASADENA, CA (US); Seyed Ali HAJIMIRI, La Canada, CA (US)

(21) Appl. No.: 13/208,314

(22) Filed: Aug. 11, 2011

Related U.S. Application Data
(60) Provisional application No. 61/373,208, filed on Aug. 12, 2010.

(51) Int. Cl. C12Q 1/02; C12M 1/38; C12M 1/34

(52) U.S. Cl. 435/29; 435/287.1; 435/286.1

(57) ABSTRACT

The present disclosure describes cell-based sensors. Cell-based sensors can comprise cells coupled with a sensor for sensing change of configuration and/or movement of the cells. Such changes of configuration and/or movement of the cells can be sensed through changes to one or more parameters such as electrical, mechanical and/or optical parameters. By way of example, the sensors can be magnetic based sensors or electrochemical sensors.

### CROSS REFERENCE TO RELATED APPLICATIONS

The present application claims priority to U.S. Provisional Application No. 61/373,208, filed on Aug. 12, 2010, which is incorporated herein by reference in its entirety.

### FIELD

The present disclosure relates to cell based sensors. Moreover, it relates to devices and methods for detecting substances.

### BACKGROUND

As one of the highly invested Research & Development fields in biotechnology industry, chemical screening is crucial for a multitude of applications, such as drug development, toxicity studies, clinical screening, point of care diagnostics, chemical toxin detection, environmental sensors and defense applications such as detection of chemical toxins or biological pathogens. A cell-based chemical screening platform can be used to provide highly reliable and sensitive testing results. Moreover, high throughputs and high scalability are particularly important for the platform if a large number of conditions are under test or the effect of a given condition/chemical for a variety of cell types must be known. Current chemical screening and/or detection devices use sensors based on chemical reaction, optical detectors (e.g. fluorescence-based), spectroscopic sensors or mass spectrometry-based sensors. Each of these modalities can have several disadvantages in detection speed and manufacturing cost, which can reduce their overall practicality for new sensing applications.

### SUMMARY

According to a first aspect, a sensing system is described, the sensing system comprising: one or more cells that change configuration and/or move as a result of presence of substances, change in environment, or intrinsic physiological change; and an individual sensing unit or an array of sensing units, each sensing unit comprising at least one sensor, the at least one sensor being coupled with the one or more cells such that the change of configuration and/or movement of the one or more cells changes the one or more electrical and/or mechanical parameters of the at least one sensor as a function of the change of configuration and/or movement of the one or more cells.

According to a second aspect, a sensing method is described, the method comprising: providing one or more cells that change configuration and/or move as a result of presence of substances, environmental change or intrinsic physiological change, wherein the substances are at or near the one or more cells; coupling one or more sensors with the one or more cells, the one or more cells changing one or more electrical and/or mechanical parameters of the one or more sensors as a function of a change of configuration and/or movement of the one or more cells; applying an analyte whereby presence of the substances is unknown to the one or more cells; and detecting the change of the one or more electrical and/or mechanical parameters of the one or more sensors as a function of a change of configuration and/or movement of the one or more cells, whereby the detected change of the one or more electrical and/or mechanical parameters corresponds to the presence or absence of the substances, environmental changes and/or physiological changes in the analyte.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIGS. 1A-1C show a CMOS circuit comprising an exemplary inductive magnetic sensor arrangement.

FIG. 2 shows an inductive capacitive resonant circuit with and without magnetic particles.

FIG. 3 shows a drawing of the exemplary inductive quad-core magnetic sensor arrangement. The dimensions are shown to provide an example scale but other dimensions are also possible.

FIG. 4 shows a block diagram of an exemplary electronic arrangement for a quad-core frequency shift magnetic sensor system.

FIG. 5 shows results of a simulated location-dependent sensor frequency shift response for a single magnetic particle.

FIG. 6 shows an exemplary frequency response graph associated with positioning and/or moving of the magnetic particles associated with the cell.

FIGS. 7A-7B show exemplary frequency response graphs for a cell before and after applying an analyte to the cell.

FIG. 8 shows an exemplary diagram of an electrochemical sensor with a cell moving and/or repositioning thereby changing the impedance between two electrodes.

FIG. 9 shows an exemplary block diagram of a two-dimensional sensor array.

FIG. 10 shows an exemplary a substance screening substrate based on cell autonomous migration and integrated magnetic sensor array.

### APPENDIX

Appendix A are enclosed herewith and form an integral part of the specification of the present application.

### DETAILED DESCRIPTION

Cell-based sensors based on electronic microcircuits present an alternative to optical and/or spectroscopic systems. Cell-based sensing makes use of biological and cellular behaviors that already exist in a given cell type and therefore allow the sensing platform to be used in a variety of contexts with an array of different cell types, as described in reference [1]. For example, the cells can be obtained from differentiation of stem cells to different cell lineages, each of which can be sensitive to a series of chemical agents and placed on the chip. In another example, the cells can be of homogenous type (e.g. fibroblasts), but move in controlled routes (e.g. circular loops) on a surface of the chip. The motion or number of cycles can be used as an output of cellular response. Yet in another example, embryonic stem cells can be differentiated into a specific cell lineage and this cell type is then placed at a location on the chip.

For example, embryonic stem cells of a mouse can be differentiated towards the cardiac lineage by formation of embryoid bodies, formed in media lacking the stimulatory hormone Leukemia Inhibitory Factor (LIF), as described in reference [2]. The sensor surface can be coated with a ligand for cell attachment. Cells can then be placed on the sensor surface and differentiated to cardiomyocytes on the surface of the sensors. The resulting cells will beat in a periodic manner, similar to a rhythmic motion of a heart muscle.

If a given set of chemicals have adverse effects on the function of the heart cells, the sensor can be configured to detect such effects. Either changes in the position and/or movement (e.g. amplitude or frequency of beating) of the cells can then be measured as a set of criteria to determine toxicity. The same platform can be used to test the effects of ion channel blockers or to screen chemical libraries for toxicity, effects, or modulate ion channel functions, as described in reference [3]. By way of example and not of limitation, the motion of a set of magnetic beads immobilized on the surface of the cardiac differentiated cells can be used to detect changes in the cell beating patterns.

The sensing platform is inherently label-free and substantially eliminates expensive and bulky imaging systems. The use of such device and methods can provide a useful approach for chemical detection, particularly for cell-based chemical detection. The term “cell” is intended to refer to any biological cell (e.g., various cells from human or animal body, plant, etc.) and/or the combination of cells with the same and/or different types. The terms “cell-based sensor” and “cell-based biological sensor” can be used interchangeably, which can be comprised of the sensing cells and the sensor instruments, such as sensor electronics. The cells act as biological sensor front-end to complement or augment stand alone sensor instruments, such as sensor electronics.

The sensing system can be built using, by way of example and not of limitation, a (complementary metal-oxide-silicon) CMOS process. A CMOS sensor can generate and detect electromagnetic signals with high accuracy and sensitivity. Moreover, it can provide unparalleled signal processing power with millions of transistors on-chip, and allow implementation of complex systems with ultra-small form factors, high reliability, and low prices.

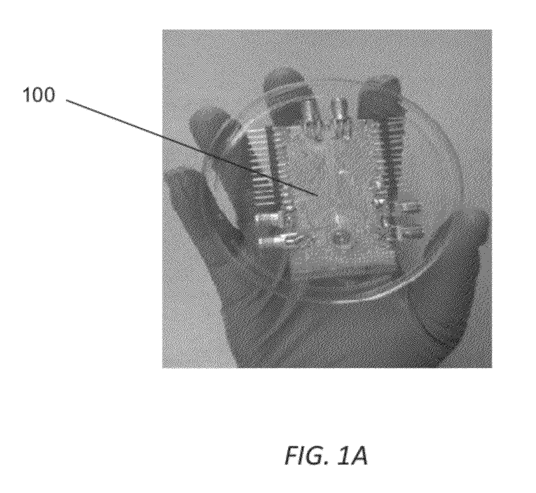

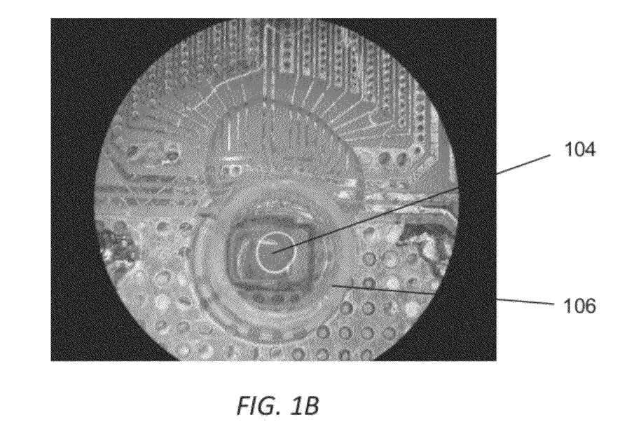

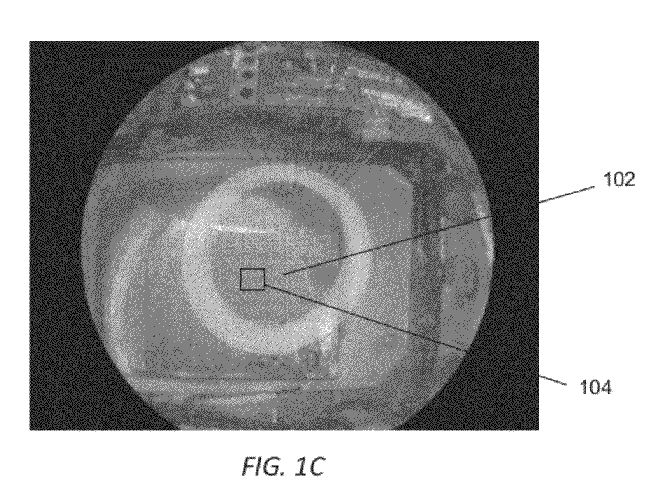

FIGS. 1A-1C show a substrate (100) with a plurality of CMOS based inductive magnetic sensors (102) (see references [8]-[12]. According to an embodiment of the present disclosure, the CMOS based inductive magnetic sensors (102) can be arranged in a plurality of sensor units (104) on the substrate (100). FIG. 1C shows a close-up view of the plurality of the sensor units (104) arranged in an array configuration and further revealing each of the inductive magnetic sensors (102) on the substrate (100). A microfluidic reservoir (106) is shown in FIG. 1B, formed around the array of the sensor units (104). The microfluidic reservoir (106) can be used to keep the culture medium for the cells. The sensor can be put into an environmental chamber, for example, for creating a high humidity environment. Furthermore, a platform comprising microfluidic channels can be fluidly communicable with the microfluidic reservoir (106) to facilitate transferring fluids. An optical detector (e.g., microscope or camera) can be placed over the microfluidic reservoir to observe and/or capture images of the cells.

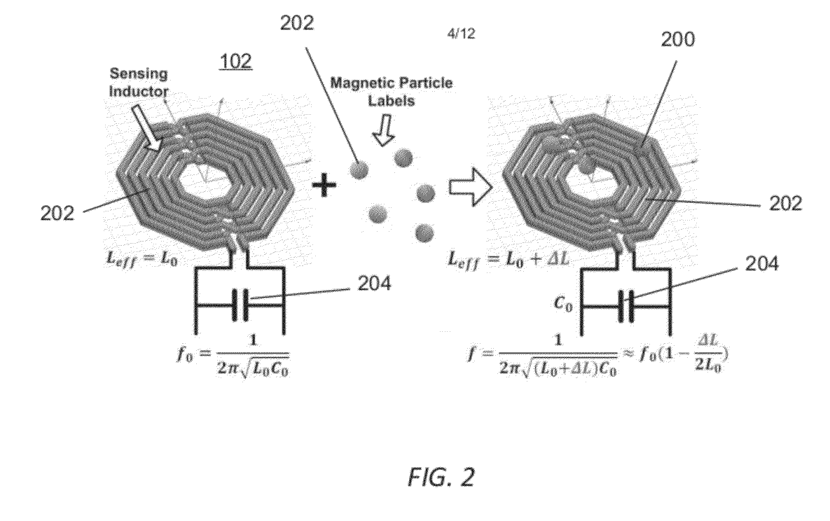

FIG. 2 shows a drawing of a single inductive magnetic sensor (102). The inductive magnetic sensor (102) is an LC resonator (e.g., inductive-capacitive tank circuit) comprised of an inductor (202) and a capacitor (204). By way of example, and not of limitation, the inductor (202) is shown as a multi-turn inductor. As known by those skilled in the art, an LC resonator has a natural resonant frequency f₀, which can be shown by the equation:

f₀=1/2π(L₀C₀)^{1/2},
where L₀ is the inductance of the inductor (202) and C₀ is the capacitance of the capacitor (204).

Current through the LC resonator generates a magnetic field, and when magnetic particles (200) are introduced on or near the inductor (202), the magnetic field polarizes the magnetic particles (200). Such polarization increases the total magnetic energy and the effective inductance of the inductor (202). The increase in the effective inductance thereby corresponds to a down-shift of the resonant frequency of the LC resonator, which can be shown as:

$$\text{?} - \frac{1}{2 π \sqrt{LC}} - \frac{1}{2 π \sqrt{( {L}_{0} + Δ L ) {C}_{0}}} \text{?} \text{?} ( 1 - \frac{Δ L}{2 {L}_{0}} ) \text{?} \\ \text{?} \text{indicates text missing or illegible when filed} \qquad ( 1 )$$

where ΔL represents the increase in the inductance due to the magnetic particles (200). Thus, the down-shift in the resonant frequency indicates the existence of magnetic particles on the surface of the sensor. Such magnetic particles can be made of various magnetic materials such as iron oxide (e.g., maghemite or magnetite).

According to some embodiments, the magnetic sensing system can be utilized to determine physiological changes to cells by sensing changes in configuration and/or movement of the cells. Such changes in configuration and/or movement of the cells can be detected by the magnetic sensing system by coating the cells (e.g., cardiomyocyte cells) with magnetic particles (200) and placing the cells on the inductive magnetic sensors (102). Therefore, as the cells change configuration and/or move as a consequence of physiological changes, the magnetic particles coated on the cells also move and/or reposition. Such movement and/or repositioning of the magnetic particles change electrical parameters, such as the induced magnetic field on the inductor (202). The change in inductance changes the resonant frequency of the inductive magnetic sensors.

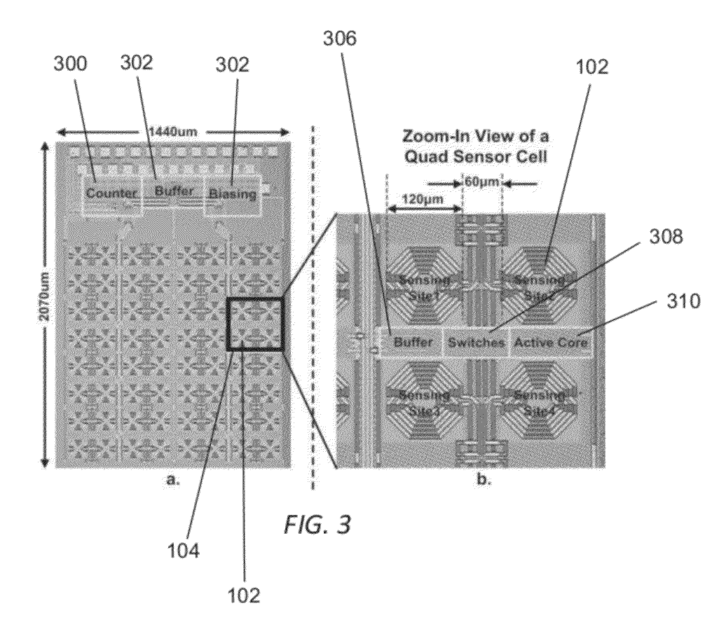

According to some embodiments, thermal stability and frequency sensitivity of the sensing device can be increased by way of a Correlated-Double-Counting (CDC) method (see references [10][11]). FIG. 3 shows an exemplary array arrangement of the CMOS inductive magnetic sensors (102) in a quad-core configuration to implement CDC. For example, four inductive magnetic sensors (102) can be arranged to form one quad-core sensing unit (104) in the quad-core configuration. By way of example and not of limitation, 16 sets of the quad-core sensing units (104) can be arranged in an array configuration to provide a total of 64 individual inductive magnetic sensors (102). In each of the quad-core sensing units (104), one of the four inductive magnetic sensors can be used as a reference sensor, thus leaving the remaining three inductive magnetic sensors to function as comparative (active) sensors by comparing the signals of the three inductive magnetic sensors against the reference sensor. Note that for a given sensing site, the role of being a comparative (active) sensor or a reference sensor can be inter-changed. In a more general implementation (e.g., N-core CDC system), at least one sensing site can be used as the reference sensor. In addition, the method of comparative sensing is for noise/drift cancellation to improve the sensitivity. Therefore, for certain low-sensitivity applications, all sensing sites can be used as the active sensor (e.g., without a reference sensor).

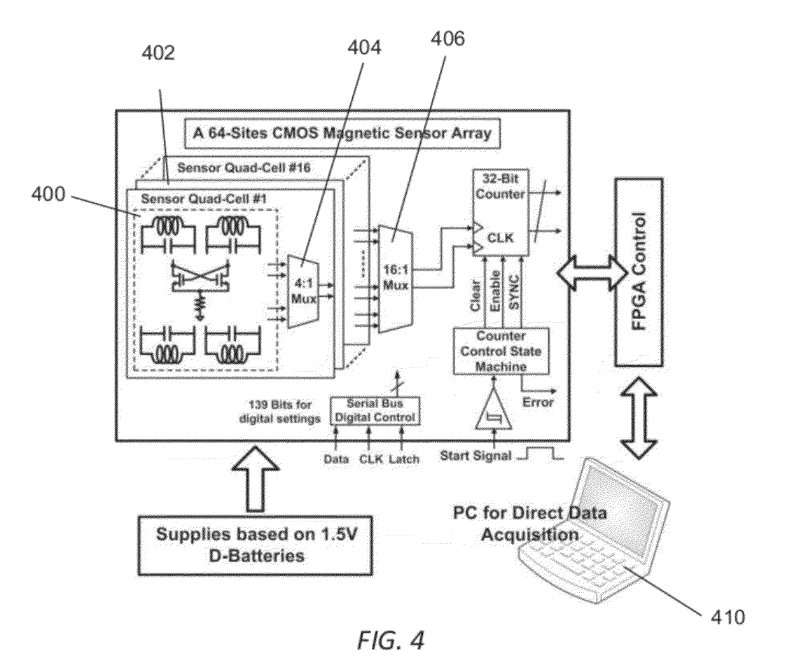

FIG. 4 shows a block diagram of an exemplary electronic arrangement of the quad-core, 64 inductive magnetic sensor, cell-based frequency shift magnetic sensor system. 16 units (402) of quad-core (400) sensing units comprising inductive magnetic sensors are shown, which can be multiplexed by a 4:1 multiplexer (404). Each sensing unit (402) of the quad-core sensors are further multiplexed by a 16:1 multiplexer (406), which can ultimately be outputted to a computer (410) for data analysis. Such electronic arrangement can be implemented, for example, in a CMOS platform as shown as a counter (300), buffer (302)(306), biasing (304), switch (308) and/or active core (310) modules in FIG. 3. The buffer (302) module can buffer the electrical signal from the inductive magnetic sensors, amplify such signals, and drive subsequent circuits. The switch (308) module can select the desired sensing unit (108) and/or the specific inductive magnetic sensor (102) to be used for sensing. The active core (310) module can comprise the core circuits which can be used with the LC sensing resonators. The biasing (304) module can provide bias for the inductive magnetic sensors, and the counter (300) module can be used to determine the resonance frequency, e.g., the output of the desired sensing unit and perform the analog-to-digital conversion of the electrical signal.

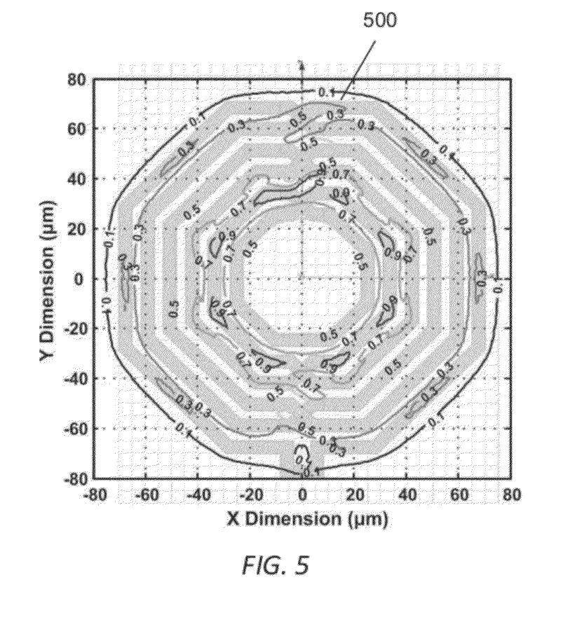

FIG. 5 shows an exemplary frequency-shift response simulation result for a 140 μm diameter inductive magnetic sensor comprising 6-turns, where a single 1 μm magnetic particle was placed at different locations of the inductive magnetic sensor. For example, when the magnetic particle is placed at the upper portion of the outermost ring (500), the simulated frequency-shift response resulted in 0.1. The frequency-shift response is shown as Δf/f₀ in ppm. Although only a 6-turn inductor is shown in the present example, different numbers of turns are possible to obtain different frequency-shift responses.

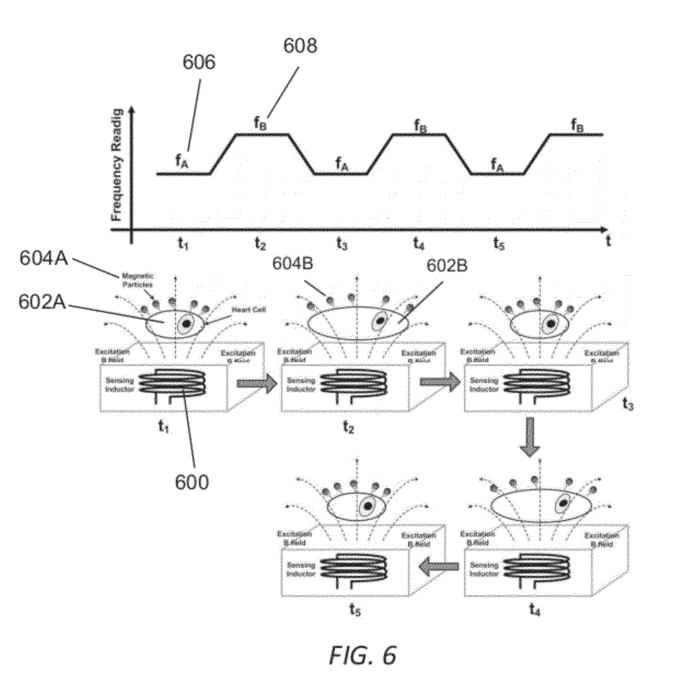

FIG. 6 shows the shift in the resonant frequency of the LC resonator (600) of the inductive magnetic sensor as the shape of a heart cell (602A)(602B) fluctuates in size over time. For example, at time t₁, the heart cell (602A) coated with magnetic particles (604A) rests on the inductive magnetic sensor such that the resonant frequency of the LC resonator (600) is f_{A} (606). At time t₂, the heart cell (602B) expands, thereby causing the magnetic particles (604B) coating the cell to move and/or reposition. The moving and/or the repositioning of the magnetic particles (604B) cause the resonant frequency of the LC resonator (600) to shift to frequency f_{B} (608). As the heart cells continue to beat, and thereby change configuration and/or move on the inductive magnetic sensor, the resonant frequency continues to fluctuate between a consistent f_{A} and f_{B}.

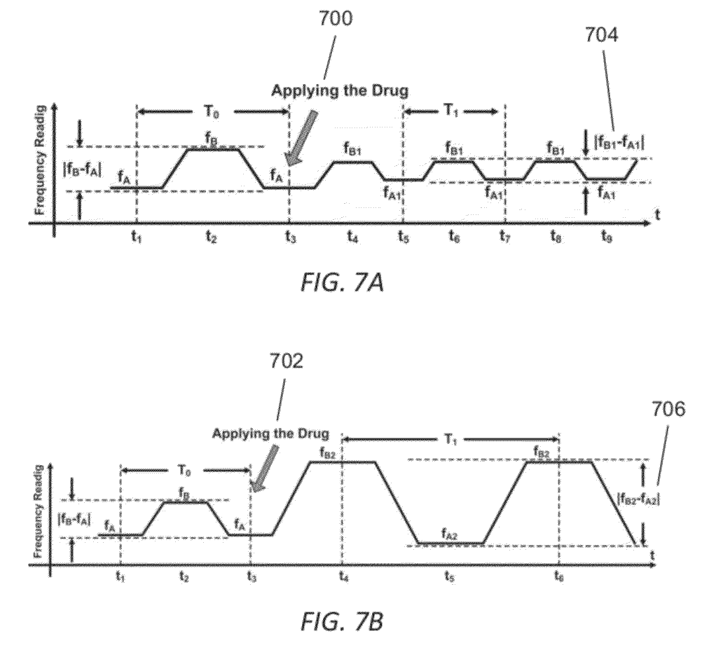

If an analyte containing a substance such as toxins is applied to the heart cell, the toxin in the analyte can cause the heart cell to change shape. This change can depend on the type of toxin that is applied. Additionally, changes to the environment or intrinsic physiological changes can also cause the cell the change configuration and/or move. FIG. 7A shows a graph for a case where the toxin that is applied (700) to the heart cell causes the magnetic particles to move in such a way that causes the change in resonant frequency to become smaller (704). FIG. 7B shows a graph for a case where the toxin that is applied (702) to the heart cell causes the magnetic particles to move in such a way that causes the change in resonant frequency to be larger (706).

Similarly, cells of any types can be placed on the inductive magnetic sensors. If an analyte is applied to the cells, then the cell's physiological behavior can change, thereby moving and/or changing configuration of the cells on the surface of the inductive magnetic sensor. Such movement and/or configuration change of the cell can cause the coated magnetic particles to also move and/or reposition, thereby causing the resonant frequency of the inductive magnetic sensors to change. If the applied analyte contains toxins, then the cell's physiological behavior will be different than the expected physiological behavior of the cell with a non-toxic analyte. Consequently, the expected resonant frequency change will also be different, thus allowing the user to conclude that the analyte may contain toxins.

In some cells, when an analyte containing toxins is introduced to the cell-based sensors, the cell can undergo a physical deformation, thereby causing the frequency output of the inductive magnetic sensor to be another frequency, different from the frequency before the cell deformation. Such difference in the frequency can be used to detect the presence of absence of the toxins.

Temperature of the cells can be maintained at desired temperatures or changed according to particularly desired temperature patterns, such as temperature cycling, by way of on-chip or off-chip temperature controller as described in reference [13], which is incorporated by reference in its entirety. Furthermore, the magnetic sensing method can be a label-free method.

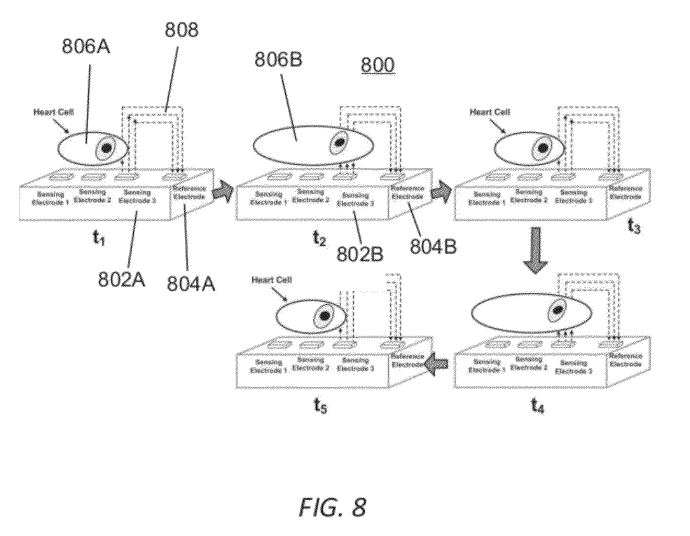

Alternatively to the inductive magnetic sensors, electrochemical sensors (800) (see references [4]-[7]) can be utilized instead, as shown in FIG. 8. In the case of electrochemical sensors (800), an effective impedance (both phase and amplitude) can be measured between a sensing electrode and a reference electrode. By way of example and not of limitation, at time t₁, a beating heart cell (806A) is in a contracted state such that a portion of the cell partially covers the path between sensing electrode 3 (802A) and the reference electrode (804A). At time t₂, the heart cell (806B) is in an expanded state such that the entire path between the sensing electrode 3 (802B) and the reference electrode (804B) is covered by the heart cell (806B), thereby resulting in a different effective impedance between the two electrodes than at time t₁. Since the heart cell is beating, the cell cycles between a contracted state and an expanded state, which can be determined by the cyclic change in the effective impedance between an electrode and the reference electrode, shown at the various time intervals in FIG. 8.

Similarly, as the physiological behavior of any type of cell is modulated by substances such as toxins, chemical, or drugs, such change in the physiological behavior can directly cause a cell to change configuration and/or move. Such change of configuration and/or movement can then be detected through the impedance measurement. Again, the temperature of the cells can be maintained at desired temperatures by way of on-chip or off-chip temperature controller as described in reference [13], which is incorporated by reference in its entirety.

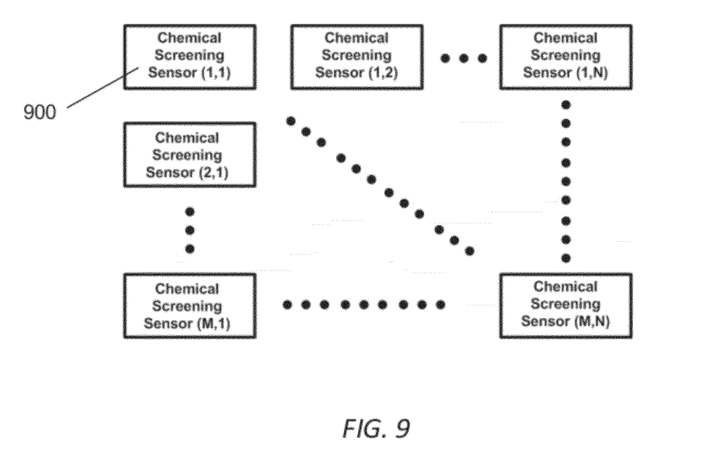

According to some embodiments, the inductive magnetic sensor and the electrochemical sensor platforms can be extended to a 1-D, 2-D, or 3-D array of sensors, thereby increasing throughput capacity and reducing the form-factor of the implementation, as shown in FIG. 9. Such array can be fabricated using CMOS technology on a single chip, multiple chips, or on a discrete basis. Each of the sensing blocks (900) represents a set of sensors configured for a desired chemical sensing application and each of the blocks (900) can be configured identically or differently. In the case where each of the blocks (900) are configured to be identical, the array of sensors allows for increased throughput configuration thereby allowing for a comprehensive study and comparison of the same type of chemical samples via different sensing methods. In the case where each of the blocks (900) are configured differently, different samples can be detected simultaneously to allow for comparison of different chemical samples with an increased throughput. Alternatively, the sensing array can be configured as a combination of same and different sensors for versatility.

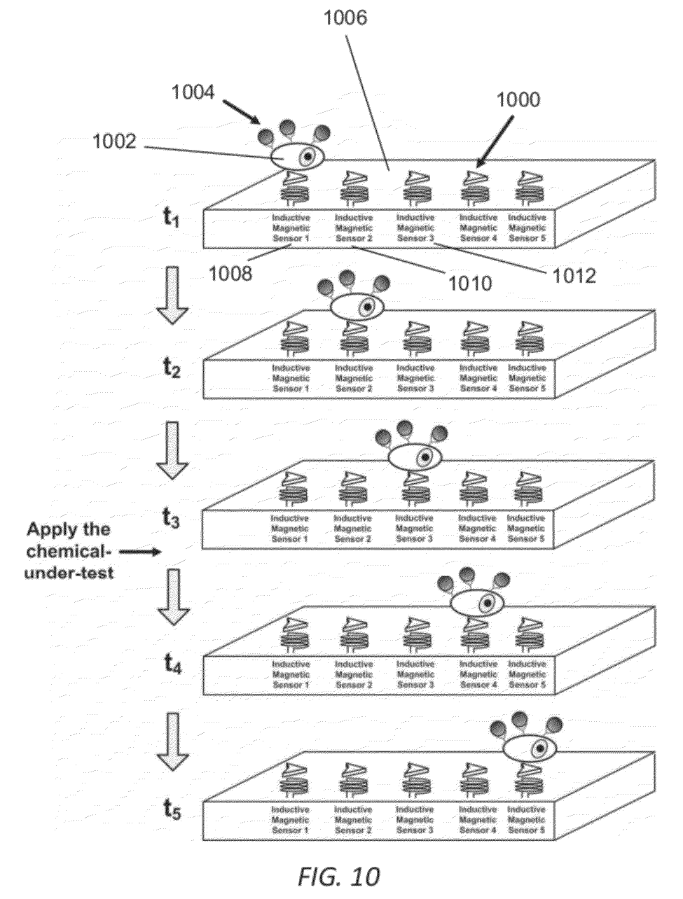

According to some embodiments, the sensor surface can be patterned with mechanical structures, and/or chemical and/or biological agent (1000) to allow for autonomous migration of the cells on the sensor surface by causing the cells to adhere or move to desired locations, as shown in FIG. 10 and also described in reference [16], which is incorporated by reference in its entirety. The distance of the migration and speeds (e.g. average and instantaneous speeds) of the cell migration can be recorded by the sensors as the cells autonomously migrate. The addition of various analytes can also affect the physiological and/or biochemical conditions of the cell environment on the surface of the sensors. Additionally, the sensor surface can be covered with glass, polymers, polydimethylsiloxane (PDMS), silicon nitride, sugars, parylene C, or other sacrificial materials, to protect the sensors from being in direct contact with the cells, thereby allowing the sensor to be implanted inside a body of flesh (e.g., human or animal body).

FIG. 10 shows a sensor platform having, by way of example and not of limitation, five inductive magnetic sensors (1006), and a cell (1002) coated with magnetic particles (1004) on a mechanically patterned, or treated with a chemical or biological agent (1000) surface. From time t₁ to t₃, the cell on the sensor surface migrates natively from inductive magnetic sensor 1 (1010) to inductive magnetic sensor 3 (1012), facilitated by the pre-patterned mechanical structures, or chemical or biological agents (1000). At time t₃, an analyte is applied to the cell on the sensor, which affects the biological condition of the cell, thus changing the migration behavior of the cell. As the treated cell continues to migrate from time t₃ to t₅, the migration behavior can be observed. Changes such as the migration speed, distance and pattern can be compared from the native migration to the migration of the chemically treated cell to recognize differences and conclude cellular behavior.

The examples set forth above are provided to give those of ordinary skill in the art a complete disclosure and description of how to make and use the embodiments of the present disclosure, and are not intended to limit the scope of what the inventors regard as their disclosure. Modifications of the above-described modes for carrying out the disclosure may be used by persons of skill in the art, and are intended to be within the scope of the following claims. All patents and publications mentioned in the specification may be indicative of the levels of skill of those skilled in the art to which the disclosure pertains. All references cited in this disclosure are incorporated by reference to the same extent as if each reference had been incorporated by reference in its entirety individually.

It is to be understood that the disclosure is not limited to particular methods or systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

A number of embodiments of the disclosure have been described. Nevertheless, it will be understood that various modifications may be made without departing from the spirit and scope of the present disclosure. Accordingly, other embodiments are within the scope of the following claims.

### LIST OF REFERENCES

1. Sharma S V, Haber D A, Settleman J. “Cell line-based platforms to evaluate the therapeutic efficacy of candidate anticancer agents.” Nat Rev Cancer. 2010 Apr;10(4):241-53. Epub 2010 Mar 19.
2. Mandavi A, Davey R E, Bhola P, Yin T, Zandstra P W. “Sensitivity analysis of intracellular signaling pathway kinetics predicts targets for stem cell fate control.” PLoS Comput Biol. 2007 Jul;3(7):e130.
3. Witchel H J. “Emerging trends in ion channel-based assays for predicting the cardiac safety of drugs.” IDrugs. 2010 Feb;13(2):90-6.
4. A. Hassibi and T. H. Lee, “A Programmable 0.18 μm CMOS Electrochemical Sensor Microarray for Biomolecular Detection,” IEEE Sensors Journal, vol 6, no.6, pp. 1380-1388, Dec. 2006.
5. M. Schienle, C. Paulus, A. Frey, F. Hofmann, B. Holzapfl, P. Schinder-Bauer, and R. Thewes, “A Fully Electronic DNA Sensor With 128 Positions and In-Pixel A/D Conversion,” IEEE J. Solid-State Circuits, vol. 39, no. 12, pp. 2438-2445, Dec. 2004.
6. F. Heer, M. Keller, G. Yu, J. Janata, M. Josowicz, and A. Hierlemann, “CMOS Electro-Chemical DNA-Detection Array with On-chip ADC,” in. IEEE ISSCC Dig. Tech. Papers, Feb. 2008, pp.168-169.
7. A. Manickam, A. Chevalier, M. McDermott, A. D. Ellington, and A. Hassibi, “A CMOS Electrochemical Impedance Spectroscopy Biosensor Array for Label-free Biomolecular Detection,” in IEEE ISSCC Dig. Tech. Papers, Feb. 2010.
8. T. Aytur, P. R. Beatty, and B. Boser, “An Immunoassay Platform Based on CMOS Hall Sensors”, Solid-State Sensor, Actuator and Microsystems Workshop, June 2002.
9. S. Han, H. Yu, B. Murmann, N. Pourmand and S. X. Wang, “A High-Density Magnetoresistive Biosensor Array with Drift-Compensation Mechanism,” in IEEE ISSCC Dig. Tech. Papers, Feb. 2007, pp.168-169.
10. H. Wang, Constantine Sideris and A. Hajimiri, “A Frequency-Shift based CMOS Magnetic Biosensor with Spatially Uniform Sensor Transducer Gain,” to appear in IEEE CICC Dig. Tech. Papers, 2010.
11. H. Wang, S. Kosai, C. Sideris, and A. Hajimiri, “An Ultrasensitive CMOS Magnetic Biosensor Array with Correlated Double Counting (CDC) Noise Suppression,” IEEE MTT-S Int. Microwave Symp. (IMS), May, 2010.
12. H. Wang, Y. Chen, A. Hassibi, A. Scherer, and A. Hajimiri, “A Frequency-Shift CMOS Magnetic Biosensor Array with Single-Bead Sensitivity and No External Magnet,” IEEE ISSCC Dig. Tech. Papers, pp.438-439, Feb. 2009.
13. H. Wang, and A. Hajimiri, “Fully Integrated Temperature Regulator for Biochemical Application”, U.S. Patent Publication 20100163545.
14. K. H. J. Buschow and F. R. de Boer, “Physics of Magnetism and Magnetic Materials”, Springer, 2003.
15. D. E. Bray and R. K. Stanley, “Nondistructive Evaluation”, Taylor and Francis Group, 1997.
16. G. Kumar, C. C. Ho, C. C. Co, “Guiding cell migration using one-way micropattern arrays”, Advance Materials, Volume 19, Issue 8, pages 1084-1090, 2007.

1. A sensing system comprising:
one or more cells that change configuration and/or move as a result of presence of substances, change in environment, or intrinsic physiological change; and
an individual sensing unit or an array of sensing units, each sensing unit comprising at least one sensor, the at least one sensor being coupled with the one or more cells such that the change of configuration and/or movement of the one or more cells changes one or more electrical and/or mechanical parameters of the at least one sensor as a function of the change of configuration and/or movement of the one or more cells.

2. The system of claim 1, wherein the one or more cells are coated with magnetic particles, the magnetic particles configured to move according to the change of configuration and/or movement of the one or more cells, the movement of the magnetic particles configured to change the one or more electrical parameters of the at least one sensor.

3. The system of claim 1, wherein the individual sensing unit or array of sensing units further comprises a reference sensor, such that a sensed output of the at least one sensor is compared with a sensed output of the reference sensor.

4. The system of claim 1, further comprising a reservoir or one or more micro-fluidic structure integrally provided on the at least one sensor such that the reservoir or the one or more micro-fluidic structure is configured to provide a culture medium for the one or more cells and the substances near the at least one sensor.

5. The system of claim 2, wherein the at least one sensor is at least one inductive magnetic sensor.

6. The system of claim 5, wherein the at least one inductive magnetic sensor is an LC resonator comprising capacitors coupled with inductors.

7. The system of claim 1, wherein the change in the one or more electrical parameters is a change in inductance.

8. The system of claim 7, wherein the change in inductance corresponds to a shift in a resonant frequency of the LC resonator.

9. The system of claim 1, wherein, in use, the one or more cells are coupled with the at least one sensor through contact.

10. The system of claim 1, wherein, in use, the one or more cells are coupled with the at least one sensor through one or more layers interposed between the one or more cells and the at least one sensor.

11. The system of claim 10, wherein the one or more layers are selected from the group consisting of: glass, polymer, sugars, PDMS, parylene C, silicon nitride, and sacrificial materials.

12. The system of claim 1, further comprising a temperature controller for maintaining the one or more cells at a temperature selected from the group consisting of: a desired temperature, set spatial temperature profile, and temporal temperature sequence.

13. The system of claim 5, wherein the at least one inductive magnetic sensor comprises four inductive magnetic sensors.

14. The system of claim 1, further comprising biological or chemical agents to cause the one or more cells to adhere or move to set locations.

15. The system of claim 1, further comprising mechanical devices to cause the one or more cells to adhere or move to set locations.

16. The system of claim 1, further comprising a detector connectable to a computer for performing analysis.

17. The system of claim 1, further comprising an electronic arrangement, the electronic arrangement comprising:
a plurality of first multiplexers, each of the first multiplexers for multiplexing sensed signals from the at least one sensor and at least a second sensor of the substance sensing units; and
a second multiplexer for multiplexing an output signal from the plurality of the first multiplexers.

18. The system of claim 1, wherein the at least one sensor is made of CMOS.

19. The system of claim 6, wherein the inductors are made of CMOS.

20. The system of claim 4, wherein the reservoir is a microfluidic reservoir comprising at least one chamber to hold test analytes and cells.

21. The system of claim 4, wherein the reservoir is fluidly communicable with microfluidic channels.

22. The system of claim 1, further comprising an optical detector for obtaining an optical image of the one or more cells.

23. The system of claim 1, wherein the system is positioned adjacent to a biological tissue.

24. The system of claim 1, wherein the at least one sensor is at least one electrochemical sensor, the electrochemical sensor comprising a plurality of electrodes.

25. The system of claim 24, wherein the change in the one or more electrical parameters is a change in impedance between at least one sensing/active electrode and at least one reference electrode, whereby the change in the impedance is a function of the change of configuration and/or movement of the one or more cells.

26. The system of claim 25, further comprising a detector to detect the change in the impedance.

27. A sensing method comprising:
providing one or more cells that change configuration and/or move as a result of presence of substances in an analyte, environmental change or intrinsic physiological change, wherein the substances are at or near the one or more cells;
coupling one or more sensors with the one or more cells, the one or more cells changing one or more electrical and/or mechanical parameters of the one or more sensors as a function of the change of configuration and/or movement of the one or more cells;
applying one or more analytes; and
detecting the change of the one or more electrical and/or mechanical parameters of the one or more sensors as a function of the change of configuration and/or movement of the one or more cells, whereby the detected change of the one or more electrical and/or mechanical parameters corresponds to the presence or absence of the substances, environmental changes and/or physiological changes in the one or more analytes.

28. The method of claim 27, further comprising coating the one or more cells with magnetic particles, the magnetic particles moving according to the change of configuration and/or movement of the one or more cells, the movement of the magnetic particles for changing the one or more electrical parameters of the one or more sensors.

29. The method of claim 28, wherein the one or more sensors are one or more inductive magnetic sensors.

30. The method of claim 29, wherein the one or more inductive magnetic sensors are LC resonators comprising capacitors coupled with inductors.

31. The method of claim 30, wherein the change in the one or more electrical parameters is a change in inductance.

32. The method of claim 31, wherein the change in inductance corresponds to a shift in a resonant frequency of the LC resonator.

33. The method of claim 27, wherein variations in temperature of the one or more cells is compensated automatically.

34. The method of claim 27, further comprising adhering or moving the one or more cells to desired positions on the one or more sensors by applying biological or chemical agents to the one or more cells.

35. The method of claim 27, further comprising adhering or moving the one or more cells to desired positions on the one or more sensors by positioning mechanical devices on the one or more sensors.

36. The method of claim 34, further comprising measuring adhesion or movement characteristics of the one or more cells as a consequence of applying the biological or chemical agents, thereby determining presence or absence of the substances in the one or more analytes.

37. The method of claim 27, wherein the coupling is performed by placing the one or more cells on the one or more sensors.

38. The method of claim 27, wherein the coupling is performed by placing the one or more sensors near the one or more cells.

39. The method of claim 27, wherein the coupling is performed through contact between the one or more cells and the one or more sensors.

40. The method of claim 27, wherein the coupling is performed through one or more layers interposed between the one or more cells and the one or more sensors.

41. The method of claim 40, wherein the one or more layers are selected from the group consisting of: glass, polymer, parylene C, PDMS, sugars, silicon nitride and sacrificial materials.

42. The method of claim 27, wherein the one or more sensors are one or more electrochemical sensors.

43. The method of claim 42, wherein the one or more electrical parameters is a change in impedance between one or more sensing electrodes and one or more reference electrodes of the one or more electrochemical sensors.

44. The method of claim 43, wherein the one or more electrochemical sensors comprise at least one detector to detect the change in the impedance.

45. The method of claim 27, further comprising positioning one or more optical detection systems on or near the one or more cells for detection.
